(19) United States
(12) Patent Application Publication
(10) Pub. No.: US 2017/0101614 A1
(43) Pub. Date: Apr. 13, 2017

(54) METHOD AND APPARATUS FOR PRODUCING ACRYLAMIDE

(71) Applicant: Mitsubishi Rayon Co., Ltd., Chiyoda-ku, Tokyo (JP)

(72) Inventors: Makoto KANO, Kanagawa (JP); Takamitsu KARIYA, Kanagawa (JP)

(73) Assignee: Mitsubishi Rayon Co., Ltd., Chiyoda-ku, Tokyo (JP)

(21) Appl. No.: 15/317,292

(22) PCT Filed: Jun. 2, 2015
(86) PCT No.: PCT/JP2015/002798

§ 371 Date: Dec. 8, 2016

(30) Foreign Application Priority Data
Jun. 12, 2014 (JP) 2014-121049

(51) Int. Cl. C12M 1/00; C12M 1/34; C12P 13/02

(52) U.S. Cl. CPC C12M 23/58; C12P 13/02; C12M 41/44

(57) ABSTRACT

Provided is a technique which can easily realize the retention time of the reaction mixture in the reactor suitable for the production quantity by controlling the amount of the reaction liquid in accordance with the production quantity and thus can suppress the amount of a biocatalyst used in a method for producing acrylamide from acrylonitrile by using a biocatalyst. Provided is a method for producing acrylamide from acrylonitrile through a continuous reaction using a biocatalyst in reactors by using two or more reactors connected in series, wherein one reactor A and a reactor B connected to the reactor A on an upstream side are communicated with each other below liquid faces of reaction liquids in both reactors, and the producing method comprises controlling a liquid volume of a reaction liquid in the reactor B by controlling a level of a reaction liquid in the reactor A to be between a disposed position of a communicating port with the reactor B and a full level position.

### TECHNICAL FIELD

The present invention relates to a method and an apparatus for producing acrylamide from acrylonitrile by using a biocatalyst.

### BACKGROUND ART

The method for producing an intended compound by utilizing a biocatalyst has advantages that the reaction condition is mild, the purity of the reaction product is high as the by-products are little, and the producing process can be simplified. In the production of an amide compound, nitrile hydratase of an enzyme to convert a nitrile compound into an amide compound is widely used since the biocatalyst was found.

As a method for industrially producing acrylamide by utilizing a biocatalyst, a so-called continuous reaction is widely used in which the produced acrylamide is continuously or intermittently taken out from the reactor without taking out the entire amount of an aqueous solution thereof while continuously or intermittently supplying a raw material and a biocatalyst into a reactor.

As a method for continuously producing acrylamide by utilizing a biocatalyst, for example, there is a method in which the liquid volume in the reactor is fixed in a constant volume, the raw material and the biocatalyst are supplied into the reactor at a constant flow rate, and the produced acrylamide aqueous solution is taken out from the reactor at a constant flow rate (see Patent Literatures 1 to 3). In addition, a method is described in Patent Literature 4 in which the liquid volume in the reactor is fixed in a constant volume and the flow rate of the raw material and the biocatalyst supplied into the reactor and the flow rate of the aqueous acrylamide solution taken out from the reactor are changed.

### PRIOR ART PUBLICATION

### Patent Publication

Patent Publication 1: JP 2001-340091 A

Patent Publication 2: WO 2012/039407 A

Patent Publication 3: WO 2009/113654 A

Patent Publication 4: WO 2010/038832 A

### SUMMARY OF THE INVENTION

### Problems to be Solved by the Invention

Industrially, the production quantity of acrylamide is changed in accordance with the demand. In the methods described in Patent Publications 1 to 4 in which the amount of the reaction liquid by the continuous reaction is fixed in a constant amount, the retention time of the reaction mixture in the reactor is changed as the production quantity of acrylamide is changed. In other words, the retention time of the reaction mixture in the reactor decreases as the production quantity of acrylamide increases, and the retention time of the reaction mixture in the reactor increases as the production quantity of acrylamide decreases in contrast. The activity of the catalyst decreases as the retention time increases since the biocatalyst contained in the reaction mixture time-dependently deteriorates. As a result, a more amount of catalyst is used in order to compensate for the decreased catalytic activity for the production of acrylamide.

On the other hand, the time for the reaction between the catalyst and acrylonitrile of the substrate decreases when the retention time of the reaction mixture in the reactor decreases, and thus a more amount of catalyst is used in order to compensate for the decreased reaction time for the production of an intended amount of acrylamide. It is industrially disadvantageous that the retention time is long or short since the amount of catalyst used in order to obtain the intended amount of acrylamide and the producing cost of acrylamide increases as a result.

In addition, even in a case in which the production quantity of acrylamide is not changed or the change is minor for a long period of time, it is rare that the retention time of acrylamide in the reactor to the production quantity thereof is an optimum time from the viewpoint of decreasing the amount of a biocatalyst used even when the amount of the reaction liquid by the continuous reaction is fixed in a constant amount.

Furthermore, a method to adjust the amount of the reaction liquid in the reactor by respectively installing a supply pump to send the raw material to each reactor or a discharge pump to take out the reaction liquid from the reactor in order to change the retention time of the reaction mixture at the time of the continuous reaction in accordance with the production quantity is not industrially preferable since not only the operation is complicated but also the equipment cost greatly increases.

Accordingly, a main object of the invention is to provide a technique which can easily realize the retention time of the reaction mixture in the reactor suitable for the production quantity by controlling the amount of the reaction liquid in accordance with the production quantity and thus can suppress the amount of a biocatalyst used in a method for producing acrylamide from acrylonitrile by using a biocatalyst.

### Means for Solving Problems

In order to solve the above problems, the invention provides the following [1] to [8].

[1] A method for producing acrylamide from acrylonitrile through a continuous reaction using a biocatalyst in reactors by using two or more reactors connected in series, wherein

one reactor A and a reactor B connected to the reactor A on an upstream side are communicated with each other below liquid faces of reaction liquids in both reactors, and

the producing method includes controlling a liquid volume of a reaction liquid in the reactor B by controlling a level of a reaction liquid in the reactor A to be between a disposed position of a communicating port with the reactor B and a full level position.

[2] The producing method according to [1], in which

the reactor A includes a circulating line to circulate a reaction liquid and a discharge line to discharge a reaction liquid, and

a level of a reaction liquid in the reactor A is controlled by adjusting a liquid volume of a reaction liquid to be discharged from the reactor A and/or a liquid volume of a reaction liquid to return to the reactor A through circulation.

[3] The producing method according to [1] or [2], in which a liquid volume of a reaction liquid in one or more other reactors located on an upstream side is controlled by controlling a level of a reaction liquid in a reactor located the most downstream among the two or more reactors.

[4] The producing method according to any one of [1] to [3], in which a liquid volume of a reaction liquid in one or more other reactors located on an upstream side is from 0.9-fold to 1.2-fold a liquid volume of a reaction liquid in a reactor located the most downstream among the two or more reactors.

[5] An apparatus for producing acrylamide from acrylonitrile through a continuous reaction using a biocatalyst in reactors, the apparatus including:

two or more reactors connected in series;

a detecting unit to detect a level of a reaction liquid in a reactor A; and

a control unit to adjust a liquid volume of a reaction liquid to be discharged from the reactor A and/or a liquid volume of a reaction liquid to return to the reactor A through circulation, in which

one reactor A and a reactor B connected to the reactor A on an upstream side have a communicating port disposed below liquid faces of reaction liquids in both reactors.

[6] The producing apparatus according to [5], in which the control unit receives an input of a signal from the detecting unit and adjusts a liquid volume of a reaction liquid to be discharged from the reactor A and/or a liquid volume of a reaction liquid to return to the reactor A through circulation to control a level of a reaction liquid in the reactor A to be between a disposed position of a communicating port with the reactor B and a full level position.

[7] The producing apparatus according to [5] or [6], in which

the reactor A includes a circulating line to circulate a reaction liquid and a discharge line to discharge a reaction liquid, and

the control unit is a pump or valve provided to the discharge line and/or the circulating line.

[8] The producing apparatus according to any one of [5] to [7], in which the communicating port is a connecting port of a line to connect reactors or a void or gap of a partition wall to partition reactors.

In addition, the invention provides the following [9] to [14] in another aspect.

[9] A method for producing acrylamide from acrylonitrile by using a biocatalyst, in which

a liquid volume in one or more reactors located on an upstream side is controlled by controlling an amount of a reaction liquid in a reactor located on a downstream side of two or more connected reactors.

[10] The method for producing acrylamide according to [9], in which amounts of reaction liquids in one or more reactors located on an upstream side are controlled by controlling an amount of a reaction liquid in a reactor located the most downstream of two or more connected reactors.

[11] The method for producing acrylamide according to [9] or [10], in which a reactor that is located on a downstream side and controls an amount of a reaction liquid includes one or more circulating lines of a reaction liquid and one or more sending lines of a reaction liquid, and an amount of a reaction liquid in a reactor located on an upstream side is controlled by adjusting a sent flow rate in the sending line.

[12] The method for producing acrylamide according to [11], in which a reactor that is located on a downstream side and controls an amount of a reaction liquid includes a device to detect a height of a liquid face of a reaction liquid and adjusts a sent flow rate of a reaction liquid in accordance with the height of the liquid face.

[13] The method for producing acrylamide according to any one of [9] to [13], in which a liquid volume of a reaction liquid in one or more reactors that are located on an upstream side and have a controlled amount of a reaction liquid is from 0.9-fold to 1.2-fold a liquid volume in a reactor that is located on a downstream side and controls an amount of a reaction liquid.

[14] An apparatus for producing acrylamide by using a biocatalyst, the apparatus including a plurality of reactors, in which

the respective reactors are connected to one another by a pipe or a void portion or gap portion for partition, and

a reactor located on a downstream side includes a circulating line to circulate a reaction liquid to another reactor and a sending line to take out a reaction liquid from a reactor.

In the present specification, the term “upstream side” refers to a side on which a reactor to which a reaction raw material (including acrylonitrile, water, and biocatalyst) is first added is located in the arrangement direction of the reactors connected in series. The upstream side or downstream side means the relative positional relation among the reactors.

### Effects of the Invention

According to the producing method of the invention, in a method for producing acrylamide from acrylonitrile by using a biocatalyst, it is possible to suppress the amount of the catalyst used by controlling the amount of the reaction liquid in the reactor and it is possible to easily produce acrylamide at low cost.

### BRIEF DESCRIPTION OF DRAWINGS

FIG. 1 is a schematic diagram illustrating an embodiment of the apparatus to be used in the method for producing acrylamide of the invention.

### MODE(S) FOR CARRYING OUT THE INVENTION

Hereinafter, preferred embodiments of the invention will be described with reference to the accompanied drawing. Incidentally, the embodiments to be described below are merely an example of representative embodiments of the invention, and the scope of the invention is thus not narrowly interpreted by this.

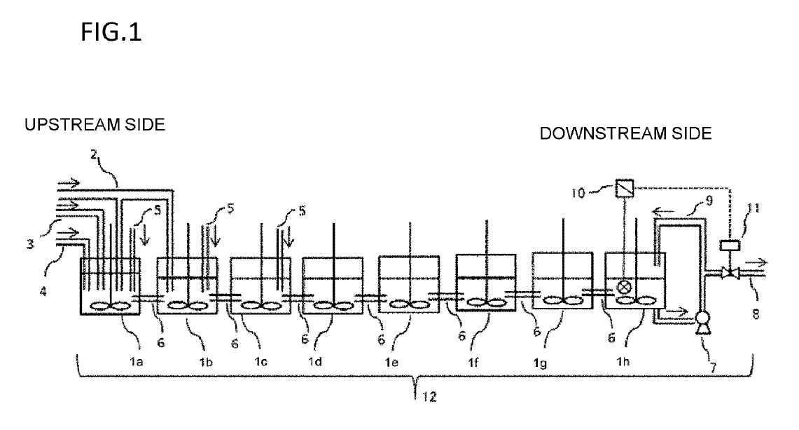

The method for producing acrylamide according to the invention is a reaction (so-called continuous reaction) in which the raw material (including acrylonitrile, water, and biocatalyst) is continuously or intermittently supplied into the reactor and the reaction mixture (hereinafter, also referred to as the “reaction liquid”) in the reactor is continuously or intermittently taken out without withdrawing the entire amount. A preferred embodiment of the apparatus to be used in the method for producing acrylamide according to the invention is illustrated in FIG. 1. A continuous reaction apparatus 12 is equipped with two or more reactors (reactor 1a to 1h) connected in series and produces acrylamide from acrylonitrile and water through a continuous reaction using a biocatalyst in each reactor. Specifically, in the continuous reaction apparatus 12, the raw material to react is first added to a reactor 1a located the most upstream and a reactor 1b connected to this to initiate the reaction and the reaction is allowed to proceed while sequentially transferring the reaction liquid to the reactor located on the downstream side. Thereafter, the reaction liquid containing acrylamide thus produced is recovered from a reactor 1h located the most downstream.

The number of reactors is not particularly limited, and it can be appropriately selected depending on the reaction conditions and the like. For example, the number of reactors is preferably from 2 to 12, more preferably from 2 to 10, and even more preferably from 2 to 8. There may be those that are connected in parallel among the reactors if necessary. The respective reactors may be independent ones or those obtained by partitioning a large reactor into plural ones by a partition wall. The respective spaces divided by the partition wall is regarded as one reactor in the case of a reactor partitioned by a partition wall.

The type of reactor is not particularly limited, and, for example, it is possible to use reactors of various types such as a stirring type, a fixed bed type, a fluidized bed type, a moving bed type, a tower type, and a pipe type. Among these, a stirring type which can promote the dispersion and mixing of the raw material is preferable. It is also possible to connect reactors of different types in combination.

A stirring blade is preferable as a stirring device. The shape of the stirring blade is also not particularly limited, and examples thereof may include a paddle, a disk turbine, a propeller, a helical ribbon, an anchor, and the Pfaudler.

Acrylonitrile is supplied into the reactor 1a located the most upstream and the reactor 1b connected to the downstream thereof through an acrylonitrile supply line 2. In addition, water and the catalyst are supplied into the reactor 1a through a water supply line 3 and a catalyst supply line 4, respectively. The reference numeral 5 denotes the alkali adding line to the reactors 1a, 1b, and 1c.

The supply of raw materials is not limited to the reactor 1a located the most upstream, and the raw material can also be supplied into the reactor located downstream thereof (for example, reactor 1b).

The kind of acrylonitrile is not particularly limited, and commercially available ones can be used. It is preferable to use acrylonitrile having a cyanide concentration of 3 ppm or less in order to decrease the amount of the biocatalyst used.

Water (raw material water) is one that is used for hydration of acrylonitrile when producing acrylamide. Examples of the water may include pure water; and an aqueous solution in which an acid, a salt, or the like is dissolved in water. Examples of the acid may include phosphoric acid, acetic acid, citric acid, boric acid, acrylic acid, and formic acid. Examples of the salt may include a sodium salt, a potassium salt, and an ammonium salt of the acids. Specific examples of the water may include water such as pure water, ultrapure water, or city water; and a buffer such as a Tris buffer, a phosphate buffer, an acetate buffer, a citrate buffer, or a borate buffer, but water is not limited thereto. The pH (20° C.) of raw material water is preferably from 5 to 9.

The biocatalyst includes an animal cell, a plant cell, a cell organelle, a bacterial cell (viable cell or dead body) containing an enzyme which catalyzes the intended reaction, or a treated product thereof. Examples of the treated product may include a crude enzyme or purified enzyme extracted from a cell, a cell organelle, or a bacterial cell and further those obtained by immobilizing an animal cell, a plant cell, a cell organelle, a bacterial cell (viable cell or dead body), or an enzyme itself by an entrapping method, a crosslinking method, a carrier binding method, or the like.

Examples of the animal cell may include monkey cell COS-7, Vero, CHO cell, mouse L cell, rat GH3, and human FL cell. Examples of the plant cell may include tobacco BY-2 cell.

Examples of the bacterial cell may include a microorganism belonging to genus Nocardia, genus Corynebacterium, genus Bacillus, genus Pseudomonas, genus Micrococcus, genus Rhodococcus, genus Acinetobacter, genus Xanthobacter, genus Streptomyces, genus Rhizobium, genus Klebsiella, genus Enterobacter, genus Erwinia, genus Aeromonas, genus Citrobacter, genus Achromobacter, genus Agrobacterium, or genus Pseudonocardia.

These animal cells, plant cells, cell organelles, or bacterial cells include not only those of a wild-type but also those of which the gene is modified.

The entrapping method of one method for immobilization is a method to enclose a bacterial cell or enzyme in a fine lattice of polymer gel or to cover the bacterial cell or enzyme with a film of a semipermeable polymer. The crosslinking method is a method to crosslink an enzyme with a reagent having two or more functional groups (polyfunctional crosslinking agent). The carrier binding method is a method to bind an enzyme to a water-insoluble carrier. Examples of the immobilizing carrier to be used in immobilization may include glass beads, silica gel, polyurethane, polyacrylamide, polyvinyl alcohol, carrageenan, alginic acid, agar, and gelatin.

Examples of the enzyme may include nitrile hydratase produced by the microorganism described above and the like.

It is possible to add a water-soluble monocarboxylate having two or more carbon atoms into the reaction liquid. The timing to add the water-soluble monocarboxylate is not particularly limited, and it is also possible to add the water-soluble monocarboxylate into the reactor located on the most upstream side so as to be contained in the reaction liquid of each reactor as the water-soluble monocarboxylate contained in the reaction liquid is transferred to the downstream side together with the reaction liquid. In addition, the water-soluble monocarboxylate may be added into each reactor before or after the reaction is initiated.

It is possible to improve the stability of acrylamide in the reaction liquid by adding a water-soluble monocarboxylate having two or more carbon atoms thereto.

The water-soluble monocarboxylate may be either of a saturated monocarboxylate or an unsaturated monocarboxylate. Examples of the saturated carboxylic acid may include acetic acid, propionic acid, and n-caproic acid. Examples of the unsaturated carboxylic acid may include acrylic acid, methacrylic acid, and vinylacetic acid. Examples of the salt may include a sodium salt, a potassium salt, and an ammonium salt of a saturated monocarboxylic acid or an unsaturated monocarboxylic acid. These water-soluble monocarboxylates may be used singly, or two or more kinds thereof may be used concurrently.

The amount of the water-soluble monocarboxylate added is preferably from 20 to 5000 mg/kg as an acid with respect to acrylamide to be produced.

The pH for the reaction to produce acrylamide through hydration of acrylonitrile is preferably from 6 to 9 and more preferably from 7 to 8.5. There are an indicator method, a metal electrode method, a glass electrode method, and a semiconductor sensor method as the method for measuring the pH, but the measurement by a glass electrode method to be industrially widely utilized is preferable.

The reaction temperature (temperature of reaction liquid) at the time of the hydration of acrylonitrile is not particularly limited, but it is preferably from 10 to 50° C., more preferably from 15 to 40° C., and even more preferably from 20 to 35° C. It is possible to sufficiently enhance the reaction activity of the biocatalyst by setting the reaction temperature to 10° C. or higher. In addition, it is possible to prevent the deactivation of the biocatalyst by setting the reaction temperature to 50° C. or lower. In addition, it is preferable to supply water or acrylonitrile to be supplied by setting the temperature thereof to be lower than the reaction temperature by 5° C. or higher in order to decrease the heat removal load of the reactor.

The reactor 1a and the reactor 1b are connected to each other by a connecting pipe 6, the communicating ports of the connecting pipes 6 in the reactor 1a and the reactor 1b are disposed so as to be located below the liquid faces of the reaction liquids in the respective reactors. In the same manner, the reactors 1b to 1g are connected to the downstream reactors 1c to 1h thereof by a connecting pipe 6, respectively.

The position of the communicating port of the connecting pipe 6 is preferably the position at 70% or less where the bottom face of the reactor in the height direction is 0% and the top face of the reactor is 100%. The adjustment range of the amount of the reaction liquid fitted to a change of the production quantity is widened by having the position at 70% or less.

As the aspect of the connection among the reactors, it is also possible to employ an aspect in which a partition wall to partition the reactor is provided and the reaction liquid is allowed to flow via the void or gap provided to the partition wall in addition to an aspect in which the independent reactors are connected to one another by the connecting pipe 6 so that the reaction liquid is able to flow therethrough. In this case, the void or gap corresponds to the communicating port of the connecting pipe 6, and the respective voids or gaps are disposed so as to be located below the liquid face of the reaction liquid in the reactors.

In the most downstream reactor 1h, a liquid height detecting device 10 to detect the level of the reaction liquid in the reactor is disposed. In addition, to the reactor 1h, a discharge line 8 to discharge the reaction liquid to the outside and a circulating line 9 to circulate the reaction liquid into the reactor 1h are joined. The discharge line 8 is branched off from the circulating line 9. The reference numerals 7 and 11 represent a pump provided to the circulating line 9 and a discharged flow rate adjusting device provided to the discharge line 8, respectively. The discharged flow rate adjusting device 11 may be a valve to be usually used. The discharged flow rate adjusting device 11 receives the output of a signal from the liquid height detecting device 10 and controls the discharged amount and circulated amount of the reaction liquid from the reactor 1h.

As the liquid height detecting device 10, it is possible to use a metal pipe type level meter, a float type level meter, a pressure type level meter, an ultrasonic level meter, a microwave level meter, or the like.

The discharge line 8 may be an independent line or a line that is branched off from the circulating line 9 as illustrated in the drawing. A pump can be utilized for the discharge of the reaction liquid. As the kind of pump, it is possible to utilize a non-positive displacement pump such as a centrifugal pump, an axial flow pump, or a mixed flow pump or a positive displacement pump such as a rotary pump or a reciprocating pump.

In the continuous reaction apparatus 12, the discharged flow rate adjusting device 11 receives the output of a signal from the liquid height detecting device 10 and adjusts the liquid volume of the reaction liquid to be discharged from the reactor 1h and/or the liquid volume of the reaction liquid to return to the reactor A through circulation to control the level of the reaction liquid in the reactor 1h to be between the disposed position of the communicating port of the connecting pipe 6 and the full level position. This makes it possible to arbitrarily control the liquid volume of the reaction liquid in the reactors 1a to 1g located on the upstream side of the reactor 1h in the continuous reaction apparatus 12.

As a preferred aspect, the liquid volume in one or more reactors located on the upstream side is controlled by controlling the level of the reaction liquid in the reactor 1h located the most downstream. It is more preferable to control the liquid volume in all the reactors located on the upstream side.

The pressure in the reaction liquid increases in proportion to the distance from the liquid face (depth of reaction liquid). The pressure of the reaction liquid by the liquid depth is equal at the disposed positions of the communicating ports of the adjacent reactors on the upstream side and the downstream side by disposing the communicating port of the connecting pipe 6 so as to be located below the liquid faces of the reaction liquids in the respective reactors, and the depth from the liquid face of the reaction liquid to the disposed position of the communicating port is thus equal in the downstream reactor and the upstream reactor.

By adjusting the height of the liquid face of the reaction liquid in the reactor located on the downstream side, it is possible to match the height of the liquid face of the reaction liquid in the reactor located on the upstream side to the height of the liquid face of the reaction liquid in the reaction liquid located on the downstream side.

It is preferable to decrease the pressure loss since the height of the liquid face of the reaction liquid in the reactor located on the upstream side is higher than the height of the liquid face of the reaction liquid in the reactor located on the downstream side by the height of the liquid face corresponding to the head loss in a case in which the pressure loss in the connecting pipe 6 is too great.

As a method to decrease the pressure loss in the production on an industrial scale, for example, a method to adjust the inner diameter of the connecting pipe 6 is considered, but the specific size of the inner diameter can be appropriately selected depending on the size of the reactor, the position (distance from liquid face of reaction liquid) of the connecting pipe 6, or the like.

For example, the inner diameter of the connecting pipe 6 is preferably from 5 to 150 mm and more preferably from 10 to 100 mm in a case in which the reaction is conducted by connecting reactors having a volumetric capacity of about from 5 to 10 L. By setting the inner diameter to 5 mm or more, it is possible to suppress the pressure loss in the connecting pipe 6 and to prevent that the liquid face of the reaction liquid in the reactor located on the upstream side increases and the reaction liquid overflows the reactor. By setting the inner diameter to 150 mm or less, it is possible to suppress the cost of the piping material. Incidentally, it means that the inner diameter is preferably from 5 to 150 mm as the corresponding diameter in a case in which the shape of the connecting pipe 6 is not circular. The inner diameter or position of each connecting pipe 6 may be the same as or different from one another in a case in which three or more reactors are connected by the connecting pipe 6. They can be appropriately selected depending on the reaction conditions and the like.

By having the position of the connecting portion (communicating port of connecting pipe 6 or void or gap of partition wall) between the reactor located on the upstream side and the reactor located on the downstream side at the position to be lower than the liquid face at all times and adjusting the inner diameter of the connecting portion, the liquid faces of the reaction liquids of the reactor located on the upstream side and the reactor located on the downstream side have the same height and the pressure caused by the liquid depth can be equalized. It is possible to control the liquid volume of the reaction liquid in the reactor located on the upstream side by controlling the level of the reaction liquid in the most downstream reactor by this. Furthermore, it is easy to control the liquid volume in the downstream reactor to the desired liquid volume by decreasing the pressure loss in the connecting pipe 6.

As described above, it is possible to arbitrarily control the liquid volume of the reaction liquid in the reactors 1a to 1g located on the upstream side of the reactor 1h in the continuous reaction apparatus 12. Hence, in the continuous reaction apparatus 12, it is possible to easily realize the retention time of the reaction liquid in the reactor suitable for the production quantity by controlling the amount of the reaction liquid in accordance with the production quantity and this makes it possible to suppress the amount of the biocatalyst used.

Incidentally, the retention time of the reaction liquid (reaction time) is not limited, but it is preferably from 1 to 30 hours and more preferably from 2 to 20 hours. Here, the retention time is a value obtained by dividing the total volumetric capacity [m³] of the reaction liquids (sum of amounts of reaction liquids in all the reactors) by the flow rate [m³/hr] of the reaction mixture to be continuously taken out from the reactor. In addition, the amount of the biocatalyst used can be appropriately selected depending on the kind and form of the biocatalyst to be used. For example, the activity of the biocatalyst to be supplied to the reactor is preferably about from 50 to 500 U per 1 mg of dry cells at a reaction temperature of 10° C. The unit U (unit) in the present specification means to produce 1 micromole of acrylamide from acrylonitrile for 1 minute.

It is preferable that the amount of the reaction liquid in each reactor located on the upstream side is from 0.9-fold to 1.2-fold the liquid volume of the reaction liquid in the reactor 1h. By setting the amount to 0.9-fold or more, it is possible to increase the volumetric capacity of the reactor and to obtain a sufficient reaction time. In addition, by setting the amount to 1.2-fold or less, it is possible to prevent that the volumetric capacity for the reaction increases too great, the retention time of the catalyst in the reactor thus increases, and the catalyst is deactivated.

In the present embodiment, an example in which a function to receive the output of a signal from the liquid height detecting device 10 and to adjust the liquid volume of the reaction liquid to be discharged from the reactor 1h and/or the liquid volume of the reaction liquid to be circulated after the discharge is imparted to the discharged flow rate adjusting device 11 is described, but the function may be imparted to the pump 7.

### EXAMPLES

Hereinafter, the invention will be described in detail with reference to Examples and Comparative Examples. However, the invention is not limited by the following description. Incidentally, the concentration “% by mass” of an aqueous acrylamide solution is simply noted as “%” in some cases.

### Example 1

### Adjustment of Biocatalyst

The Rodococcus rhodochrous J1 strain exhibiting nitrile hydratase activity (deposited at the National Institute of Advanced Industrial Science and Technology, International Patent Organism Depositary (Central 6, 1-Banchi, Higashi 1-Chome, Tsukuba, Ibaraki Prefecture, Japan) as the accession number FERM BP-1478 on Sep. 18, 1987) was aerobically cultured in a medium (pH: 7.0) containing glucose at 2%, urea at 1%, peptone at 0.5%, yeast extract at 0.3%, and cobalt chloride hexahydrate at 0.01% (all of them represent % by mass) at 30° C. This was harvested and washed by using a centrifuge and a 0.1% aqueous solution of sodium acrylate (pH: 7.0), thereby obtaining a bacterial cell suspension (dry bacterial cell: 15% by mass).

(Reaction from Acrylonitrile to Acrylamide)

As the reactor, 4 pieces of stirrers equipped with jacket cooling (inner diameter: 18 cm, height: 26 cm, inner volumetric capacity: 6.6 L) were connected in series. For the connection of each reactor, a SUS pipe (with gate valve) having an inner diameter of 15 mm was attached to the position at a distance of 5 cm from the bottom face of the reactor. To each reactor, 4 pieces of inclined paddle wings (angle of inclination: 45°, blade diameter: 8 cm) were disposed. The reactors were denoted as the first reactor, the second reactor, the third reactor, and the fourth reactor from the reactor on the upstream side into which the raw material was supplied, and the most downstream reactor from which the reaction liquid was taken out to the outside was denoted as the fourth reactor. A circulating line to return the reaction liquid to the fourth reactor by a pump was provided to the reactor outlet of the fourth reactor.

In addition, the circulating line was branched off so as to install a discharge line to take out the reaction liquid to the outside of the reactor. A valve to adjust the flow rate of the reaction liquid to be taken out was installed to the discharge line to take out the reaction liquid. An ultrasonic level meter was installed to the fourth reactor, and the liquid face meter and the flow rate adjusting valve installed to the discharge line were interlocked so as to be able to arbitrarily control the liquid volume of the reaction liquid in the fourth reactor. A pH control meter was installed to each reactor so as to be able to arbitrarily control the pH of the reaction liquid.

In the present Example, the desired concentration of the aqueous acrylamide solution to be taken out from the reactor was set to 50% or more.

(Production Quantity of Acrylamide: 40 kg/Day)

(1) The valve of the connecting pipe to link the reactors is closed.

(2) Aqueous acrylamide solutions having a concentration of 35%, 45%, 50%, and 50% were introduced into the reactors of from the first reactor to the fourth reactor by 4 L, respectively.

(3) The bacterial cell suspension was added into from the first reactor to the fourth reactor by 10 g, respectively.

(4) The valve of the connecting pipe to link the reactors is opened.

(5) Raw material water (pH: 7.0), acrylonitrile, and the bacterial cell suspension were continuously supplied into the first reactor at 2040 g/hr, 750 g/hr, and 12 g/hr, respectively, only acrylonitrile was continuously supplied into the second reactor at 500 g/hr, and the continuous reaction was initiated under the condition in which the production quantity of acrylamide was set to 40 kg/day. During the continuous reaction, a 1% aqueous solution of sodium hydroxide was added into each reactor so that the pH of the reaction liquid was 7.0.

(6) The amount of the reaction liquid in the fourth reactor was controlled to be 4 L by interlocking the liquid face meter and the flow rate adjusting valve of the discharge line to take out the reaction liquid.

The temperature was controlled by using cooling water (5° C.) in the jacket so that the temperature of the reaction liquid in from the first reactor to the fourth reactor was 20, 21, 22, and 23° C., respectively.

In one day after the continuous reaction was initiated, the acrylamide concentration in the reaction liquid to flow out through the discharge line of the fourth reactor was measured by using a refractometer (ATAGO RX-7000α). Acrylamide at 50.5% of the intended acrylamide concentration was detected.

Next, a reaction was conducted in the same manner as in the reaction except that only the supply of the bacterial cell suspension was changed to 10 g/hr and the amount of the reaction liquid in the fourth reactor was controlled to be 6 L by interlocking the liquid face meter and the flow rate adjusting valve of the discharge line to take out the reaction liquid.

In one day after the reaction condition was changed, the acrylamide concentration in the reaction liquid to flow out through the discharge line of the fourth reactor was measured by using the refractometer. Acrylamide at 50.6% of the intended acrylamide concentration was detected.

After the measurement of acrylamide concentration, the supply of the raw material to all the reactors was stopped, and the pump of the circulating line and taking out of the reaction liquid through the discharge line were stopped, and the valve of the connecting pipe of each reactor was closed. The entire amount of the reaction liquid in each reactor was withdrawn and the volume thereof was measured by using a measuring cylinder, and the volume of the reaction liquid present in the respective reactors of the first reactor, the second reactor, the third reactor, and the fourth reactor was 6.2 L, 6.1 L, 6.0 L, and 5.9 L, respectively.

### Comparative Example 1

Acrylamide was produced from acrylonitrile in the same manner as in Example 1 except that an overflow pipe (made of SUS having an inner diameter of 15 mm) was installed at the position at a distance of 16 cm from the bottom face of each reactor so that the liquid volume of the reaction liquid was 4 L instead of installing the connecting pipe to the reactor and the reaction liquid was sent to the downstream reactor through the overflow pipe and the reaction liquid was taken out to the outside of the reactor through the overflow pipe of the fourth reactor.

In the same manner as in Example 1, in one day after only the supply of the bacterial cell suspension was changed to 10 g/hr, the acrylamide concentration in the reaction liquid to flow out through the overflow pipe of the fourth reactor was measured. Acrylamide at 46.2% lower than the intended acrylamide concentration was detected.

In the same manner as in Example 1, the amount of the reaction liquid in each reactor was measured, and the volume of the reaction liquid present in the respective reactors of the first reactor, the second reactor, the third reactor, and the fourth reactor was 4.2 L, 4.1 L, 4.0 L, and 3.9 L, respectively.

### Example 2

### Production Quantity of Acrylamide: 80 kg/Day

The same reactor as in Example 1 was used.

(1) The valve of the connecting pipe to link the reactors is closed.

(2) Aqueous acrylamide solutions having a concentration of 35%, 45%, 50%, and 50% were introduced into the reactors of from the first reactor to the fourth reactor by 6 L, respectively.

(3) The bacterial cell suspension prepared in Example 1 was added into from the first reactor to the fourth reactor by 15 g, respectively.

(4) The valve of the connecting pipe to link the reactors is opened.

(5) Raw material water (pH: 7.0), acrylonitrile, and the bacterial cell suspension were continuously supplied into the first reactor at 4090 g/hr, 1500 g/hr, and 32 g/hr, respectively, only acrylonitrile was continuously supplied into the second reactor at 1000 g/hr, and the continuous reaction was initiated under the condition in which the production quantity of acrylamide was set to 80 kg/day. During the continuous reaction, a 1% aqueous solution of sodium hydroxide was added into each reactor so that the pH of the reaction liquid was 7.0.

(6) The amount of the reaction liquid in the fourth reactor was controlled to be 6 L by interlocking the liquid face meter and the flow rate adjusting valve of the discharge line to take out the reaction liquid.

The temperature was controlled by using cooling water (5° C.) in the jacket so that the temperature of the reaction liquid in from the first reactor to the fourth reactor was 20, 21, 22, and 23° C., respectively.

In one day after the continuous reaction was initiated, the acrylamide concentration in the reaction liquid to flow out through the discharge line of the fourth reactor was measured in the same manner as in Example 1. Acrylamide at 50.5% of the intended acrylamide concentration was detected.

In the same manner as in Example 1, the amount of the reaction liquid in each reactor was measured. The volume of the reaction liquid present in the respective reactors of the first reactor, the second reactor, the third reactor, and the fourth reactor was 6.2 L, 6.1 L, 6.0 L, and 5.9 L, respectively.

### Comparative Example 2

The continuous reaction was conducted in the same manner as in Example 2 except that the same reactor as in Comparative Example 1 was used and the amount of the reaction liquid in each reactor was set to 4 L.

In one day after the continuous reaction was initiated, the acrylamide concentration in the reaction liquid to flow out through the overflow pipe of the fourth reactor was measured in the same manner as in Example 2. Acrylamide at 45.1% lower than the intended acrylamide concentration was detected.

In the same manner as in Example 1, the amount of the reaction liquid in each reactor was measured. The volume of the reaction liquid present in the respective reactors of the first reactor, the second reactor, the third reactor, and the fourth reactor was 4.2 L, 4.1 L, 4.0 L, and 3.9 L, respectively.

### Example 3

### Production Quantity of Acrylamide: 80 kg/Day

The reaction was conducted in the same manner as in Example 2 except that the temperature was controlled by using cooling water (5° C.) in the jacket so that the temperature of the reaction liquid in from the first reactor to the fourth reactor was all 38° C. and the amount of the reaction liquid in the fourth reactor was controlled to be 2 L by interlocking the liquid face meter and the flow rate adjusting valve of the discharge line to take out the reaction liquid.

In one day after the continuous reaction was initiated, the acrylamide concentration in the reaction liquid to flow out through the discharge line of the fourth reactor was measured in the same manner as in Example 1. Acrylamide at 50.3% of the intended acrylamide concentration was detected.

In the same manner as in Example 1, the amount of the reaction liquid in each reactor was measured. The volume of the reaction liquid present in the respective reactors of the first reactor, the second reactor, the third reactor, and the fourth reactor was 2.2 L, 2.1 L, 2.0 L, and 1.9 L, respectively.

### Comparative Example 3

The continuous reaction was conducted in the same manner as in Example 3 except that the same reactor as in Comparative Example 1 was used and the amount of the reaction liquid in each reactor was set to 4 L.

In one day after the continuous reaction was initiated, the acrylamide concentration in the reaction liquid to flow out through the discharge line of the fourth reactor was measured in the same manner as in Example 1. Acrylamide at 48.7% lower than the intended acrylamide concentration was detected.

In the same manner as in Example 1, the amount of the reaction liquid in each reactor was measured. The volume of the reaction liquid present in the respective reactors of the first reactor, the second reactor, the third reactor, and the fourth reactor was 4.2 L, 4.1 L, 4.0 L, and 3.9 L, respectively.

### Example 4

### Production Quantity of Acrylamide: 20 kg/Day

The same reactor as in Example 1 was used.

(1) The valve of the connecting pipe to link the reactors is closed.

(2) Aqueous acrylamide solutions having a concentration of 35%, 45%, 50%, and 50% were introduced into the reactors of from the first reactor to the fourth reactor by 2 L, respectively.

(3) The bacterial cell suspension prepared in Example 1 was added into from the first reactor to the fourth reactor by 5 g, respectively.

(4) The valve of the connecting pipe to link the reactors is opened.

(5) Raw material water (pH: 7.0), acrylonitrile, and the bacterial cell suspension were continuously supplied into the first reactor at 1020 g/hr, 375 g/hr, and 5 g/hr, respectively, only acrylonitrile was continuously supplied into the second reactor at 250 g/hr, and the continuous reaction was initiated under the condition in which the production quantity of acrylamide was set to 20 kg/day. During the continuous reaction, a 1% aqueous solution of sodium hydroxide was added into each reactor so that the pH of the reaction liquid was 7.0.

(6) The amount of the reaction liquid in the fourth reactor was controlled to be 2 L by interlocking the liquid face meter and the flow rate adjusting valve of the discharge line to take out the reaction liquid.

The temperature was controlled by using cooling water (5° C.) in the jacket so that the temperature of the reaction liquid in from the first reactor to the fourth reactor was all 30° C.

In one day after the continuous reaction was initiated, the acrylamide concentration in the reaction liquid to flow out through the discharge line of the fourth reactor was measured in the same manner as in Example 1. Acrylamide at 50.7% of the intended acrylamide concentration was detected.

In the same manner as in Example 1, the amount of the reaction liquid in each reactor was measured. The volume of the reaction liquid present in the respective reactors of the first reactor, the second reactor, the third reactor, and the fourth reactor was 2.2 L, 2.1 L, 2.0 L, and 1.9 L, respectively.

### Comparative Example 4

The continuous reaction was conducted in the same manner as in Example 4 except that the same reactor as in Comparative Example 1 was used and the amount of the reaction liquid in each reactor was set to 4 L.

In one day after the continuous reaction was initiated, the acrylamide concentration in the reaction liquid to flow out through the discharge line of the fourth reactor was measured in the same manner as in Example 1. Acrylamide at 42.0% lower than the intended acrylamide concentration was detected.

In the same manner as in Example 1, the amount of the reaction liquid in each reactor was measured. The volume of the reaction liquid present in the respective reactors of the first reactor, the second reactor, the third reactor, and the fourth reactor was 4.2 L, 4.1 L, 4.0 L, and 3.9 L, respectively.

TABLE 1
<Controlling the amount of the reaction liquid and the
acrylamide concentration in the reaction liquid taken
out from the most downstream reactor (the fourth reactor)>

| | | Amount | | |
|---|---|---|---|---|
| | Controlling the | of the | Production | |
| | amount of the | reaction | quantity of | Acrylamide |
| | reaction liquid | liquid | acrylamide | concentration |
| Example 1 | controlled | 6 L | 40 kg/day | 50.6% |
| Example 2 | controlled | 6 L | 80 kg/day | 50.5% |
| Example 3 | controlled | 2 L | 80 kg/day | 50.3% |
| Example 4 | controlled | 2 L | 20 kg/day | 50.7% |
| Comparative | without control | 4 L | 40 kg/day | 46.2% |
| Example 1 | | | | |
| Comparative | without control | 4 L | 80 kg/day | 45.1% |
| Example 2 | | | | |
| Comparative | without control | 4 L | 80 kg/day | 48.7% |
| Example 3 | | | | |
| Comparative | without control | 4 L | 20 kg/day | 42.0% |
| Example 4 | | | | |

### INDUSTRIAL APPLICABILITY

According to the producing method of the invention, it is easy to adjust the retention time of the reaction liquid since the amount of the reaction liquid can be controlled with favorable operability in a method for continuously producing acrylamide by using a biocatalyst, and it is possible to produce acrylamide at low cost by suppressing the amount of a biocatalyst used.

### EXPLANATIONS OF LETTERS OR NUMERALS

1 REACTOR
2 ACRYLONITRILE SUPPLY LINE
3 WATER SUPPLY LINE
4 CATALYST SUPPLY LINE
5 ALKALI ADDING LINE
6 CONNECTING PIPE
7 CIRCULATING PUMP
8 DISCHARGE LINE
9 CIRCULATING LINE
10 LIQUID HEIGHT DETECTING DEVICE
11 DISCHARGED FLOW RATE ADJUSTING DEVICE
12 CONTINUOUS REACTION APPARATUS

1: A method for producing acrylamide from acrylonitrile through a continuous reaction using a biocatalyst in two or more reactors connected in series, the method comprising
controlling a liquid volume of a reaction liquid in a reactor B by controlling a level of a reaction liquid in a reactor A to be between a disposed position of a communicating port with the reactor B and a full level position,
wherein
the reactor B is connected to the reactor A on an upstream side, and
the reactor A and the reactor B are communicated with each other below liquid faces of the reaction liquids in both reactors.

2: The producing method according to claim 1, wherein
the reactor A includes a circulating line to circulate the reaction liquid and a discharge line to discharge the reaction liquid, and
the level of the reaction liquid in the reactor A is controlled by adjusting a liquid volume of the reaction liquid to be discharged and/or a liquid volume of the reaction liquid to return to the reactor A through circulation.

3: The producing method according to claim 1, wherein a liquid volume of a reaction liquid in one or more other reactors located on an upstream side is controlled by controlling a level of a reaction liquid in the most downstream reactor among the two or more reactors.

4: The producing method according to claim 1, wherein a liquid volume of a reaction liquid in one or more other reactors located on an upstream side is from 0.9-fold to 1.2-fold a liquid volume of a reaction liquid in the most downstream reactor among the two or more reactors.

5: An apparatus for producing acrylamide from acrylonitrile through a continuous reaction using a biocatalyst, the apparatus comprising:
two or more reactors connected in series;
a detecting unit to detect a level of a reaction liquid in a reactor A; and
a control unit to adjust a liquid volume of a reaction liquid to be discharged from the reactor A and/or a liquid volume of a reaction liquid to return to the reactor A through circulation,
wherein
a reactor B is connected to the reactor A on an upstream side, and
the reactor A and the reactor B have a communicating port disposed below liquid faces of reaction liquids in both reactors.

6: The apparatus according to claim 5, wherein the control unit receives an input of a signal from the detecting unit and adjusts the liquid volume of the reaction liquid to be discharged from the reactor A and/or the liquid volume of the reaction liquid to return to the reactor A through circulation to control a level of the reaction liquid in the reactor A to be between a disposed position of the communicating port with the reactor B and a full level position.

7: The apparatus according to claim 5, wherein
the reactor A includes a circulating line to circulate the reaction liquid and a discharge line to discharge the reaction liquid, and
the control unit is a pump or valve provided to the discharge line and/or the circulating line.

8: The apparatus according to claim 5, wherein the communicating port is a connecting port of a line to connect reactors or a void or gap of a partition wall to partition the reactors.

9: The producing method according to claim 2, wherein a liquid volume of a reaction liquid in one or more other reactors located on an upstream side is controlled by controlling a level of a reaction liquid in the most downstream reactor among the two or more reactors.

10: The producing method according to claim 2, wherein a liquid volume of a reaction liquid in one or more other reactors located on an upstream side is from 0.9-fold to 1.2-fold a liquid volume of a reaction liquid in the most downstream reactor among the two or more reactors.

11: The producing method according to claim 3, wherein a liquid volume of a reaction liquid in one or more other reactors located on an upstream side is from 0.9-fold to 1.2-fold a liquid volume of a reaction liquid in the most downstream reactor among the two or more reactors.

12: The apparatus according to claim 6, wherein
the reactor A includes a circulating line to circulate the reaction liquid and a discharge line to discharge the reaction liquid, and
the control unit is a pump or valve provided to the discharge line and/or the circulating line.

13: The apparatus according to claim 6, wherein the communicating port is a connecting port of a line to connect reactors or a void or gap of a partition wall to partition the reactors.

14: The apparatus according to claim 7, wherein the communicating port is a connecting port of a line to connect reactors or a void or gap of a partition wall to partition the reactors.
